(19) United States
(12) Patent Application Publication
(10) Pub. No.: US 2003/0119814 A1
(43) Pub. Date: Jun. 26, 2003

(54) Novel prodrugs of N-H bond-containing compounds and methods of making thereof

(75) Inventors: Victor R. Guarino, Lawrence, KS (US); Veranja Karunaratne, Lawrence, KS (US); Valentino J. Stella, Lawrence, KS (US)

Correspondence Address: Jean M. Dickman, SHOOK, HARDY & BACON L.L.P., 1200 Main Street, Kansas City, MO, 64105-2118, US

(73) Assignee: The University of Kansas

(21) Appl. No.: 10/272,330

(22) Filed: Oct. 16, 2002

Related U.S. Application Data
(60) Provisional application No. 60/329,868, filed on Oct. 16, 2001.

(51) Int. Cl. A61K 31/55; C07C 381/00; A61K 31/4015; A61K 31/445

(52) U.S. Cl. 514/212.01; 514/315; 514/424; 514/665; 514/646; 540/604; 546/184; 548/542; 564/75

(57) ABSTRACT

The present invention relates to novel prodrugs of pharmaceutical compounds containing one or more N—H bonds. More specifically, the present embodiment of the invention relates to prodrugs wherein sulfur-containing promoieties are attached to pharmaceutical compounds which contain one or more N—H bonds to produce prodrugs containing at least one N—S bond. These N—S bond-containing prodrugs could have optimized stability, solubility, cell membrane permeability, pharmacokinetic properties and other pharmaceutical properties over the pharmaceutical compounds from which they are formed, depending upon the nature of the promoiety. Reversion of the prodrug to the parent pharmaceutical compound occurs by the reaction of the prodrugs with thiol molecules such as cysteine, glutathione or any other thiol containing molecule. Further, the present invention relates to methods of making N—S bond-containing prodrugs of pharmaceutical compounds containing one or more N—H bonds whereby sulfur-containing promoieties are attached to the parent compounds to create at least one N—S bond.

### CROSS-REFERENCE TO RELATED APPLICATIONS

This application claims the benefit of priority to Provisional Application No. 60/329,868 filed on Oct. 16, 2001.

### STATEMENT REGARDING FEDERALLY SPONSORED RESEARCH OR DEVELOPMENT

This work was supported by a training grant through the NIH, namely the National Cancer Institute, Grant Number CA 09242-24. This work was also supported by the Center for Drug Delivery Research, HBC and the University of Kansas.

### FIELD OF THE INVENTION

The present invention relates to novel prodrugs of pharmaceutical compounds containing one or more N—H bonds. More specifically, the present embodiment of the invention relates to prodrugs wherein sulfur-containing promoieties are attached to pharmaceutical compounds which contain one or more N—H bonds to produce prodrugs containing at least one N—S bond. These N—S bond-containing prodrugs could have optimized stability, solubility, cell membrane permeability, pharmacokinetic properties and other pharmaceutical properties over the pharmaceutical compounds from which they are formed, depending upon the nature of the promoiety. Reversion of the prodrug to the parent pharmaceutical compound occurs by the reaction of the prodrugs with thiol molecules such as cysteine, glutathione or any other thiol containing molecule. Further, the present invention relates to methods of making N—S bond-containing prodrugs of pharmaceutical compounds containing one or more N—H bonds whereby sulfur-containing promoieties are attached to the parent compounds to create at least one N—S bond.

### BACKGROUND OF THE INVENTION

Many pharmaceutical compounds have physicochemical properties which create barriers to attaining their maximum therapeutic potential. Under specific conditions, such compounds may be reversibly modified to overcome their undesirable properties through the creation of prodrugs. The basic concept underlying prodrug chemistry is schematically illustrated in FIG. 1. A compound's physicochemical properties may be temporarily modified by attaching a promoiety to the compound. The compound-promoiety derivative is referred to as a prodrug. In this chemically modified form of the compound, the prodrug is able to overcome the original barrier. Once the barrier is overcome, the prodrug reverts to the parent compound through a process referred to as transformation or reversion. The pharmaceutical compound is then able to interact with its appropriate receptor and elicit the intended pharmacological response at the compound's site of action.

To date, there does not appear to be any known art using technology to create N—S bond-containing prodrugs. However, Bridges studied the inactivation of toxin and carcinogen, N-trichloromethylthio-4-cyclohexane 1,2-dicarboximide or alternatively N-(trichloromethylthio)-3a,4,7,7a-tetrahydrophthalimide (“Captan”), a fungicide used for agricultural purposes and in paints, soaps, paper and leather. Captan has very low solubility and an N—S bond. Captan was administered to rabbits and the levels of Captan were tested. The study found that Captan, in its active form, was broken down in the body due to high thiols in the blood. Thus, this resulted in the rapid inactivation of Captan, thus, lowering the biological and genetic damage caused to a mammalian body by Captan. Bridges, B. A., The Mutagency of Captan and Related Fungicides. Mutation Research, 1975 32: p. 3-34.

Pharmaceutical compounds having certain functional groups which contain one or more N—H bonds, tend to be poorly water- and lipid- soluble due to their hydrogen bond forming potential. Examples of such N—H containing functional groups include, but are not limited to, imides, hydantoins, uracils, barbitals, amides, peptides, ureas, carbamates, aliphatic amines, aromatic amines, sulfonamides and N-containing heterocycles such as imidazole and pyroles. In addition to poor solubility, these functional groups also may lead to permeability problems and other poor pharmacokinetic and pharmaceutical properties including high polarity. Further, the amide and peptide bonds present in some of these functionalities are susceptible to peptidase cleavage. As a result, pharmaceutical compounds containing these functional groups, while having desirable therapeutic potential, have various barriers to achieving their full potential. Accordingly, for compounds having N—H bond-containing functionalities to achieve maximum therapeutic potential, these barriers must be overcome.

### SUMMARY OF THE INVENTION

The present invention is directed to sulfenamide prodrugs of N—H bond-containing compounds, the prodrugs having a general structure as illustrated in the following Formula I,

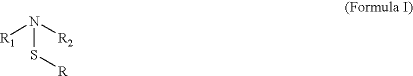

wherein R₁ and R₂ are substituents of the parent N—H bond-containing compound and R is a substituent of the sulfur-containing promoiety. R, R₁ and R₂ may be the same or different and each may be hydrogen or any organic or inorganic residue.

R may include, but is not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

R may also include, but is not limited to:

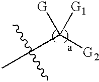

where a is an integer from 0-10 and G, G₁, and G₂, may be the same or different and are:

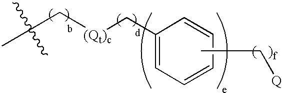

where b, c, d, e and f are integers from 0-10, Q₁ is oxygen or sulfur and Q is selected from the group comprising:

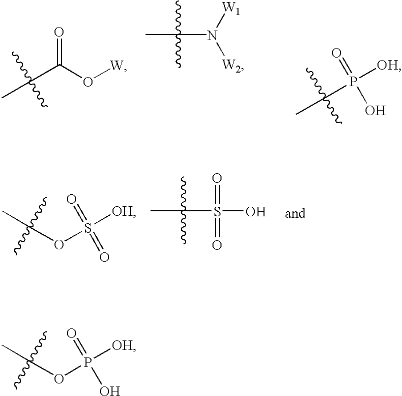

wherein W, W₁ and W₂ may be, but are not limited to,

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of R may include, but are not limited to:

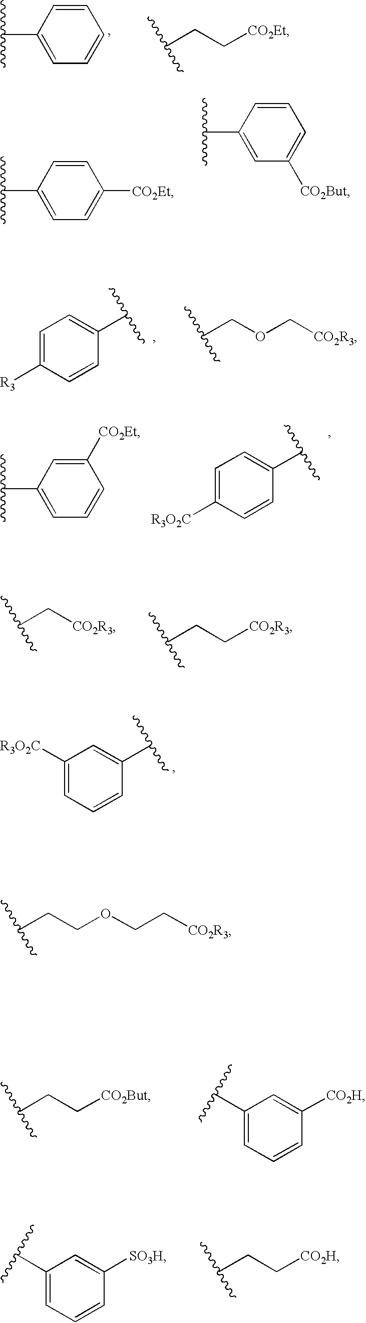

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

R₁ and R₂ may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

In another of its aspects, the invention is directed to prodrugs of pharmaceutical compounds having one or more N—H bonds, the prodrugs containing at least one N—S bond.

The present invention is further drawn to methods of making N—S bond-containing prodrugs. One method for the synthesis of prodrugs involves a derivatizing reagent of the general form represented in Formula II,

R—S—H (Formula II)

its salt represented by the general Formula III,

R—S⁻M⁺ (Formula III)

or its disulfide represented by the general Formula IV,

R—S—S—R (Formula IV)

wherein in each of Formulas II, III and IV, R may be polar or non-polar depending upon the changes desired in the properties of the parent pharmaceutical compound. R may be hydrogen or any inorganic or organic residue as described above.

Methods for the synthesis of prodrugs may alternatively involve a derivatizing reagent of the general form represented in Formula V,

R—S—X (Formula V)

wherein X may be any good leaving group including, but not limited to, halides such as chlorine or bromine. R may be hydrogen or an organic or inorganic residue and may be either polar or non-polar, as described above, depending upon the desired changes in the properties of the parent pharmaceutical compound.

In a further aspect, the present invention is directed to a first method of making novel prodrugs of N—H bond-containing compounds, the prodrug synthesis illustrated in Reaction Scheme I,

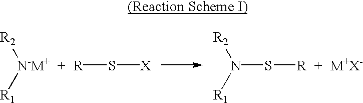

wherein M is a pharmaceutically acceptable organic or inorganic cation and X is any good leaving group such as, but not limited to, halides like chlorine or bromine. It will be understood and appreciated that the illustrated nitrogen-containing compound may be synthesized from an N—H bond-containing compound by any means known to those of skill in the art.

The present invention is further directed to a second method of making novel prodrugs of N—H bond-containing compounds, the prodrug synthesis illustrated in Reaction Scheme II,

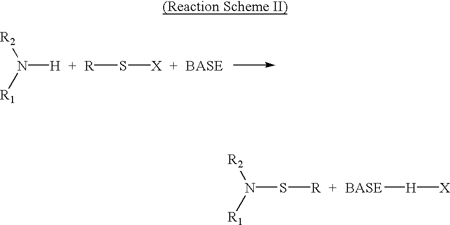

wherein X is any good leaving group such as, but not limited to, halides like chlorine or bromine.

The present invention is further directed to a third method of making novel prodrugs of N—H bond-containing compounds, the prodrug synthesis illustrated in Reaction

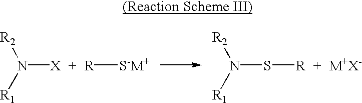

wherein M is a pharmaceutically acceptable organic or inorganic cation and X is any good leaving group such as, but not limited to, halides like chlorine or bromine. It will be understood and appreciated that the illustrated N—X bond-containing compound may be synthesized from an N—H bond-containing compound by any means known to those of skill in the art.

Still further, the present invention is directed to a fourth method of making novel prodrugs of N—H bond-containing compounds, the prodrug synthesis illustrated in Reaction Scheme IV,

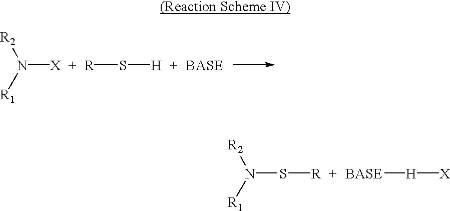

wherein X is any good leaving group such as, but not limited to, halides like chlorine or bromine. It will be understood and appreciated that the illustrated N—X bond-containing compound may be synthesized from an N—H bond-containing compound by any means known to those of skill in the art.

In each of Reaction Schemes I-IV, R₁ and R₂ are substituents of an N—H containing pharmaceutical compound. R is a substituent of a sulfur-containing promoeity. R, R₁ and R₂ may be the same or different and each may be hydrogen or any organic or inorganic residue, as described above.

Further discussion of the synthesis of sulfenamides may be found in Craine, Leslie et. al, The Chemistry of Sulfenamides, Chemical Reviews June 1989: Volume 89, Number 4 and Koval, I. V., Synthesis and Application of Sulfenamides, Russian Chemical Reviews 65 (5) 421-440 (1996) and references therein.

In another aspect, the present invention is directed to methods of using novel N—S prodrugs to optimize chemical stability, solubility, cell membrane permeability, pharmacokinetic properties and other pharmaceutical properties over the parent N—H bond-containing pharmaceutical compounds.

Additional aspects of invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means, instrumentalities and combinations particularly pointed out in the appended claims.

### BRIEF DESCRIPTION OF THE DRAWINGS

In the accompanying drawing which forms a part of the specification and is to be read in conjunction therewith:

FIG. 1 is a schematic representation of the prodrug concept.

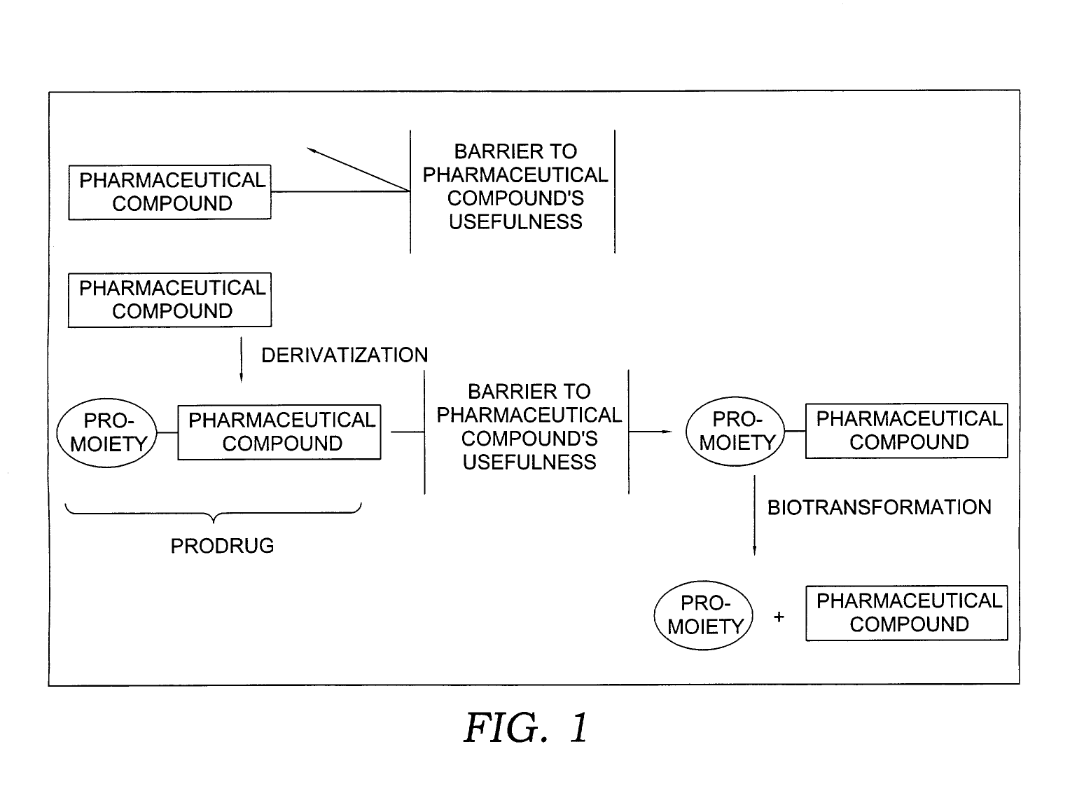

### DETAILED DESCRIPTION OF THE INVENTION

The present invention is directed to novel prodrugs of various pharmaceutical compounds containing one or more N—H bonds, as well as to methods of making and using such prodrugs. The particular embodiments described herein are intended in all respects to be illustrative rather than restrictive. Alternative embodiments will become apparent to those skilled in the art to which the present invention pertains without departing from its scope.

The present invention is drawn to sulfenamide prodrugs of pharmaceutical compounds containing one or more N—H bonds formed through a reversible derivatization to yield prodrugs with potentially optimized stability, solubility, cell membrane permeability, pharmacokinetic properties and other pharmaceutical properties, depending upon the choice of promoiety. As used herein, the term “sulfenamide” refers to the broad class of N—S bond-containing compounds, wherein the S is bivalent. Similarly, “sulfenamide prodrug(s)” refers to prodrug(s) having a N—S bond, wherein the S is bivalent. This unique utilization of N—S bond chemistry is applicable to the production of derivatives of various pharmaceutical compounds containing one or more N—H bonds and provides a general method for enhancing the chemical stability, solubility, cell membrane permeability, pharmacokinetic properties and other pharmaceutical properties of these classes of compounds to produce the pharmaceutical activity upon in vivo conversion to the parent molecule, thus facilitating their clinical use. This unique utilization of N—S bond chemistry also may provide a means for enhancing drug targeting as the reversion of these N—S bond-containing prodrugs can involve the reaction of the prodrug with free sulfhydryl containing compounds (e.g., glutathione or biological cysteine) which are present in high concentration in certain tissues or at particular sites. As such, the pharmaceutical compound may be delivered selectively to those tissues/sites rich in glutathione or other thiol-containing molecules.

The following depicts a general scheme of the reversion of a prodrug to the parent pharmaceutical compound by showing the reaction of the prodrug with thiol containing molecules. This reaction is rapid and quantitative. R¹ is a substituent of an endogenous thiol-containing molecule. R is a substituent of a sulfur-containing promoeity and is described below. R₁ and R₂ are substituents of the parent compound and also are described below.

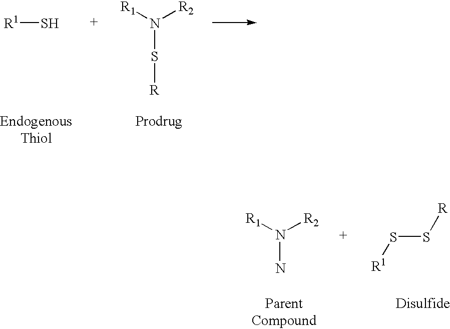

The following depicts an example the possible reversion of a prodrug to a linezolid pharmaceutical compound by showing the reaction of the prodrug with thiol containing molecules. This reaction is rapid and quantitative

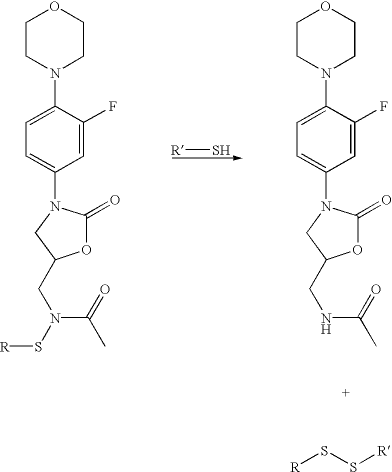

Formula I, above, depicts the general structure of sulfenamide prodrugs of the present invention wherein R₁ and R₂ are substituents of the parent compound. The remainder of the structure represents the sulfur-containing promoiety.

R, as used herein, includes, but is not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

R may also include, but is not limited to:

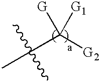

where a is an integer from 0-10 and G, G₁, and G₂, may be the same or different and are:

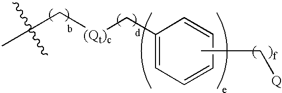

wherein b, c, d, e and f are integers from 0-10, Q₁ is oxygen or sulfur and Q is one of the following:

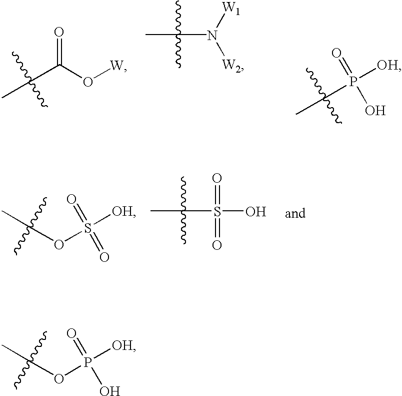

wherein, W, W₁ and W₂ may be, but are not limited to,

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of R may include, but are not limited to:

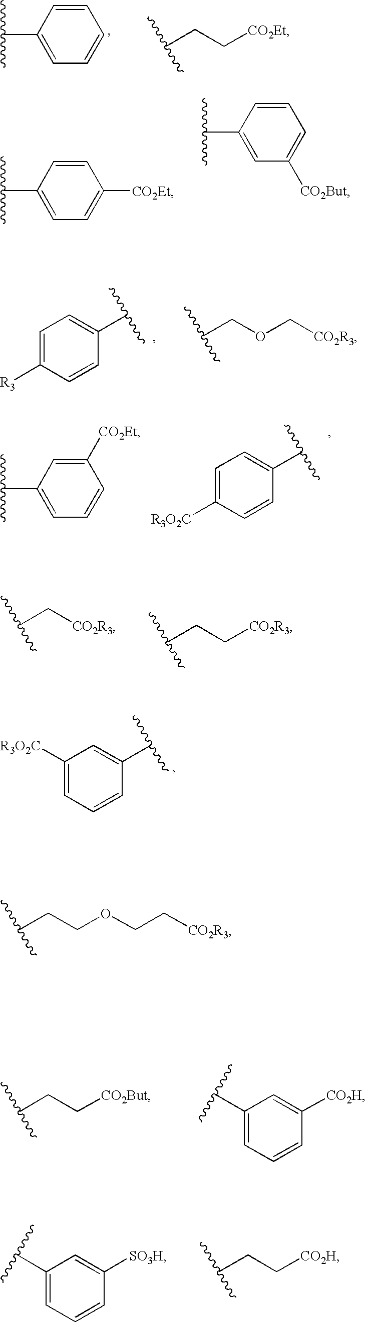

R₃, as used herein, may be a hydrogen, an inorganic residue or an organic residue. R₃ may include, but is not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Pharmaceutical compound, as used herein, includes, but is not limited to, substance(s) for the treatment and diagnosis of disease states of humans and animals. Pharmaceutical compounds containing one or more N—H bonds which are contemplated as being useful in the present invention and which maybe converted to sulfenamide prodrugs using one or more of the above-illustrated Reaction Schemes include the following:

A) Imides: Parent imide compounds, cyclic and acyclic, having the representative structure illustrated in Formula VI,

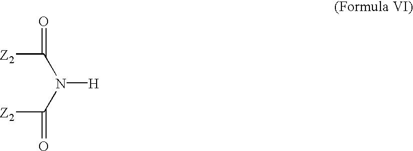

react to form sulfenamide prodrugs having a general structure as illustrated in Formula VII.

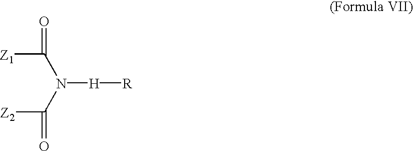

In each of Formulas VI and VII, Z₁-Z₂ are substituents of the parent imide compound. Each of R, Z₁ and Z₂ may be the same or different and each may be hydrogen or any organic or inorganic residue. R is a substituent of the sulfur-containing promoiety and is described above.

Z₁ and Z₂, as substituents of the parent imide compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of pharmaceutical compounds with this classification include, but are not limited to, ethoxsuximide, hydroorotic acid, lumazine, diflubenzuron, dantrolene, febarbamate, acetylpheneturide, biuret, acecarbromal, apronalide, carbromal, lumichrome, phenytoin, allantoin, 5-fluorouracil, mephobarbital, heptabarbital, phthalimide (model compound) and carbubarb.

B) Hydantoins. Parent hydantoin compounds have a representative structure as shown in Formula VIII.

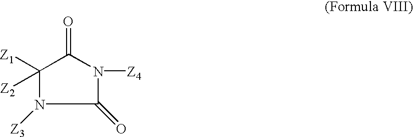

In VIII, Z₁-Z₄ are substituents of the parent hydantoin compound. Each of R, Z₁-Z₄ may be the same or different and each may be hydrogen or any organic or inorganic residue. R is a substituent of the N—S containing promoiety, as described above. However, one of Z₃ or Z, or both Z₃ and Z₄ must be hydrogen. Z₁-Z₄, as substituents of the parent hydantoin compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Compounds of the general structure of Formula VIII, react to form sulfenamide prodrugs having a general structure as illustrated in Formula IX,

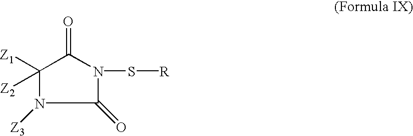

or Formula X.

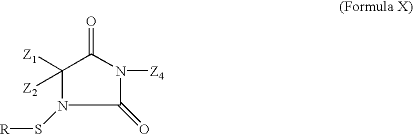

To facilitate reaction of either Z₄ to form a sulfenamide prodrug of Formula IX, or Z₃ to form a sulfenamide prodrug of Formula X, the alternative reaction site may be protected by any means known to those of skill in the art.

Examples of pharmaceutical compounds, which may be classified as hydantoins include, but are not limited to, dantrolene, ethotoin, phenytoin and allantoin.

C) Uracils. Parent uracil compounds have a representative structure as shown in Formula XI.

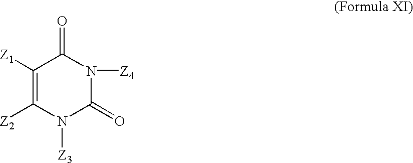

In Formula XI, Z₁-Z₄ are substituents of the parent uracil compound. Each of R and Z₁-Z₄ may be the same or different and each may be hydrogen or any organic or inorganic residue. R is a substituent of a sulfur-containing promoeity, as described above. However, one of Z₃ or Z₄, or both Z₃ and Z₄ must be hydrogen.

Z₁-Z₄, as substituents of the parent uracil compound, may include, but are not limited to,

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Compounds of the general structure of Formula XI, react to form sulfenamide prodrugs having a general structure as illustrated in Formula XII,

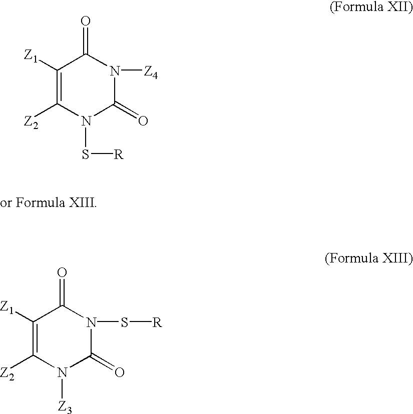

To facilitate reaction of either Z₃ to form a sulfenamide prodrug of Formula XII, or Z₄ to form a sulfenamide prodrug of Formula XIII, the alternative reaction site may be protected by any means known to those of skill in the art.

Examples of pharmaceutical compounds which may be classified as uracils include, but are not limited to, lumazine, lumichrome, 5-fluorouracil and ketanserin.

D) Barbitals. Parent barbital compounds have a representative structure as shown in Formula XIV.

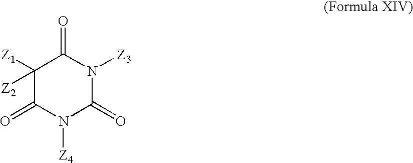

In Formula XIV, Z₁-Z₄ are substituents of the parent barbital compound. Each of R and Z₁-Z₄ may be the same or different and each may be hydrogen or any organic or inorganic residue. R is a substitutent of a sulfur-containing promoeity as described above. One of Z₃ or Z₄, or both Z₃ and Z₄ must be hydrogen.

Z₁-Z₄, as substituents of the parent barbital compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Compounds of the general structure of Formula XIV, react to form sulfenamide prodrugs having a general structure as illustrated in Formula XV,

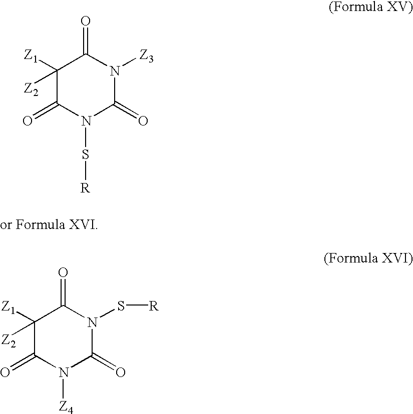

To facilitate reaction of either Z₅ to form a sulfenamide prodrug of Formula XV, or Z₆ to form a sulfenamide prodrug of Formula XVI, the alternative reaction site may be protected by any means known to those of skill in the art.

Examples of pharmaceutical compounds which may be classified as barbitals include, but are not limited to, mephobarbital and heptabarbital, febarbamate and carbubarb.

E) Amides or Peptides. Parent amide compounds having the representative structure illustrated in Formula XVII,

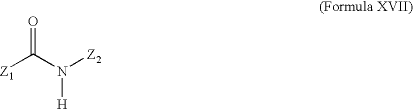

react to form sulfenamide prodrugs having a general structure as illustrated in Formula XVIII.

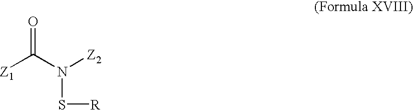

In each of Formulas XVII and XVIII, Z₁ and Z₂ are substituents of the parent amide or peptide compound. R, Z₁ and Z₂ may be the same or different and each may be hydrogen or any organic or inorganic residue. R is a substituent of the sulfur-containing promoiety, as described above.

Z₁ and Z₂, as substituents of the parent amide or peptide compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. Z₂ may be a substituted or unsubstituted acyl group, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of pharmaceutical compounds with this classification include, but are not limited to, hydroorotic acid, lumazine, diflubenzuron, biuret, apronalide, carbomal, lumichrome, phenytoin, allantoin, 5-fluorouracil, ethotoin, ketanserin, carbamazepine, nevirapine, indinavir, benzamide (model compound), acetanilide, linezolid, paclitaxel, thalidomide and any peptide drug with an available amide N—H bond.

F) Ureas: Parent urea compounds have a representative structure as shown in Formula XIX.

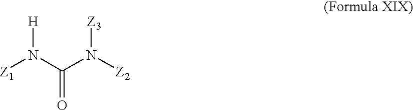

Compounds of the general structure of Formula XIX, react to form sulfenamide prodrugs having a general structure as illustrated in Formula XX.

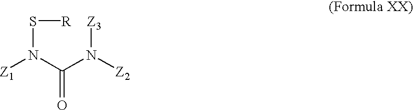

In each of Formulas XIX and XX, Z₁-Z₃ are substituents of the parent urea compound. Each of R, Z₁-Z₃ may be the same or different and each may be hydrogen or any organic or inorganic residue. R is a substituent of the sulfur-containing promoiety as described above. If either Z₂ or Z₃, or both Z₂ and Z₃ are hydrogen resulting in a compound having multiple potential reaction sites, the sites at which reaction is not desired may be protected by any means known to those of skill in the art.

Z₁-Z₃, as substituents of the parent urea compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted aromatic groups such as phenyl, benzyl, etc., with or without any additional polar or non-polar functional groups and/or heteroatoms;

4. substituted or unsubstituted cyclic groups, such as cyclohexane, etc., with or without any additional polar or non-polar functional groups and/or heteroatoms; and

5. any combination of 1), 2), 3), and 4) with or without additional polar or non-polar functional groups and/or heteroatoms.

Examples of pharmaceutical compounds with this classification, include but are not limited to: hydroorotic acid, lumazine, diflubenzuron, biuret, apronalide, carbromal, lumichrome, phenytoin, allantoin, 5-fluorouracil, ethotoin, ketanserin and carbamazepine.

G) Carbamates. Parent carbamate compounds have a representative structure as shown in Formula XXI.

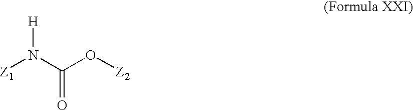

Compounds of the general structure of Formula XXI, react to form sulfenamide prodrugs having a general structure as illustrated in Formula XXII.

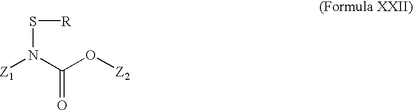

In each of Formulas XXI and XXII, Z₁ and Z₂ are substituents of the parent carbamate compound. Each of R, Z₁ and Z₂ may be the same or different and each may be hydrogen or any organic or inorganic residue. R is a substituent of the sulfur-containing promoiety, as described above.

Z₁ and Z₂, as substituents of the parent carbamate compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. Z₂ may be a substituted or unsubstituted acyl group, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of pharmaceutical compounds having this classification include, but are not limited to, febarbamate, carbubarb and felbamate.

H) Amines. Parent amine compounds may be either aliphatic or aromatic. Examples of pharmaceutical compounds which may be classified as amines include, but are not limited to dapsone, chloroquine, sulfadiazine, famciclovir, imidazoles, lasoprazole, and omeprazole.

i) Aliphatic amines. Parent aliphatic amide compounds have a representative structure as illustrated in Formula XXIII.

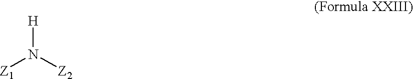

Compounds of the general structure of Formula XXIII, react to form sulfenamide prodrugs having a general structure as illustrated in Formula XXIV.

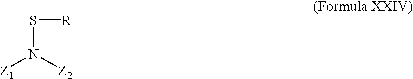

In each of Formulas XXIII and XXIV, Z₁ and Z₂ are substituents of the parent aliphatic amine compound. Each of R, Z₁ and Z₂ may be the same or different and each may be hydrogen or any organic or inorganic residue. R is a substituent of the sulfur-containing promoiety as described above.

Z₁ and Z₂, as substituents of the parent aliphatic amine compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted aromatic groups such as phenyl, benzyl, etc., with or without any additional polar or non-polar functional groups and/or heteroatoms;

4. substituted or unsubstituted cyclic groups, such as cyclohexane, etc., with or without any additional polar or non-polar functional groups and/or heteroatoms; and

6. any combination of 1), 2), 3), and 4) with or without additional polar or non-polar functional groups and/or heteroatoms.

ii) Aromatic amines. Parent aromatic amine compounds have a representative structure as illustrated in Formula XXV.

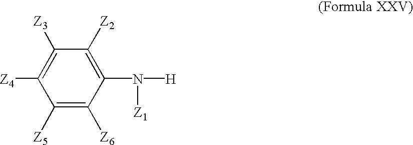

Compounds of the general structure of Formula XXV, react to form sulfenamide prodrugs having a general structure as illustrated in Formula XXVI.

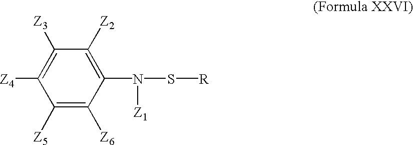

In each of Formulas XXV and XXVI, Z₁-Z₆ are substituents of the parent aromatic amine. Each of R and Z₁-Z₆ be the same or different and each may be a hydrogen or any organic or inorganic residue. R is a substituent of the sulfur-containing promoiety, as described above.

Z₁ and Z₂, as substituents of the parent aromatic amine compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. Z₂ may be a substituted or unsubstituted acyl group, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

I) N-containing heterocycles. As defined herein, parent N-containing heterocycles having the representative structure illustrated in Formula XXVII,

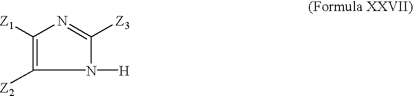

react to form sulfenamide prodrugs having a general structure as illustrated in Formula XXVIII.

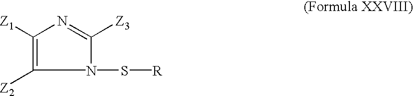

In each of Formulas XXVII and XXVIII Z₁-Z₃ are substituents of the parent aromatic amine. Each of R and Z₁-Z₃ may be the same or different and each may be a hydrogen or any organic or inorganic residue. R is a substituent of the sulfur-containing promoiety, as described above.

Z₁-Z₃, as substituents of the parent aromatic amine compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted acyl groups, with or without any additional polar or non-polar functional group and/or heteroatoms;

4. substituted or unsubstituted aromatic groups such as phenyl, benzyl, etc., with on-polar functional groups and/or heteroatoms;

5. substituted or unsubstituted cyclic groups, such as cyclohexane, etc., with or without any additional polar functional groups and/or heteroatoms; and

6. any combination of 1), 2), 3), 4) and 5) with or without additional polar or non-polar functional groups and/or heteroatoms.

Alternatively, Z₁ and Z₂ may instead take the form of an aromatic or non-aromatic ring, resulting in a parent N-containing heterocycle having a general structure as shown in Formula XXIX.

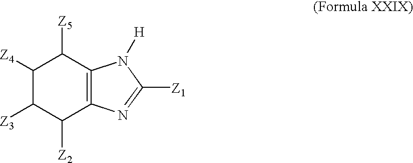

N-containing heterocycles having a general structure as shown in Formula XXIX react to form sulfenamide prodrugs having a general structure as illustrated in Formula XXX.

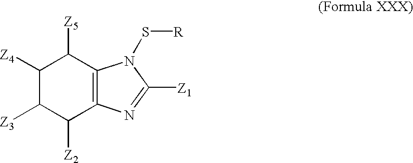

In each of Formulas XXIX-XXX Z₁-Z₅ are substituents of the parent N-containing heterocycle. Each of R and Z₁-Z₅ may be the same or different and each may be hydrogen or any organic or inorganic residue. R is a substituent of the sulfur-containing promoiety as described above

Z₁-Z₅, as substitutes of the parent N-containing heterocycle compound, may include, but are not limited to:

1. straight-chain substituted or unsubstitited aliphatic groups, such as —CH₃, CH₂CH₃, with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of pharmaceutical compounds which may be classified as N-containing heterocycles include, but are not limited to, imidazole (model compound), lansoprazole, and omeprazole.

J) Sulfonamides. Parent sulfonamides having the representative structure illustrated in Formula XXXI,

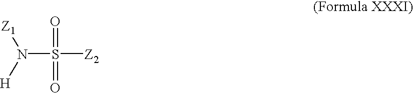

react to form sulfenamide prodrugs having a general structure as illustrated in Formula XXXII.

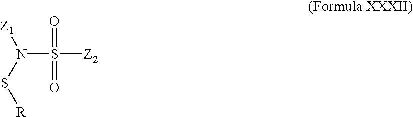

In each of Formulas XXXI and XXXII, Z₁ and- Z₂ are substituents of the parent sulfonamide compound. Each of R, Z₁ and Z₂ may be the same or different and each may be hydrogen or any organic or inorganic residue. R is a substituent of the sulfur-containing promoiety as described above.

Z₁ and Z₂, as substituents of the parent sulfonamide compound, may include, but are not limited to:

1. straight-chain substituted or unsubstituted aliphatic groups, such as —CH₃, CH₂CH₃, etc., with or without any additional polar or non-polar functional groups and/or heteroatoms;

2. branched substituted or unsubstituted aliphatic groups, with or without any additional polar or non-polar functional groups and/or heteroatoms;

3. substituted or unsubstituted acyl groups, with or without any additional polar or non-polar functional groups and/or heteroatoms;

4. substituted or unsubstituted aromatic groups such as phenyl, benzyl, etc., with or without any additional polar or non-polar functional groups and/or heteroatoms;

5. substituted or unsubstituted cyclic groups, such as cyclohexane, etc., with or without any additional polar or non-polar functional groups and/or heteroatoms; and

6. any combination of 1), 2), 3), 4) and 5) with or without additional polar or non-polar functional groups and/or heteroatoms.

Examples of pharmaceutical compounds which may be classified as sulfonamides include, but are not limited to, sulfadiazine and celecoxib.

Suitable pharmaceutical carriers for the embodiment of the present invention include any aqueous or non-aqueous carriers which are useful for administration of the drug or prodrug, preferably those which are non-toxic, otherwise inert, medically acceptable and compatible with the prodrug. Particularly useful are buffer saline based carriers. The present compositions may further comprise other active ingredients such as antimicrobial agents and other agents such as preservatives, solubilizers and stabilizers.

The present prodrug may also be in a solid form. The solid form may be a tablet, dry powder or granules. The solid form may also contain a suitable binder material, coating agent or other ingredients that facilitate formulation.

The present compositions may be administered though a variety of routes of administration. These routes include, but are not limited to, parenteral, oral, intramuscular, subcutaneous, nasal, dermal, ophthalmic, inhalation, pulmonary, vaginal, rectal and aural routes.

The following are examples of methods of making N—S bond-containing compounds according to the present invention. It will be understood and appreciated that the following examples are merely exemplary and not intended to limit the invention in any way.

### EXAMPLE 1

### Synthesis of Phthalimide-Cysteine Derivatives

Phthalimide is an N—H bond-containing imide compound and the model compound utilized for development of the present invention. Cysteine is a sulfur-containing molecule that has been determined to react with a variety of N—H bond-containing compounds, including Phthalimide. Various derivatives of cysteine have also been found to be suitable derivatizing agents for the N—S chemistry of the present invention.

The following reaction was performed entirely under anhydrous conditions and in an inert atmosphere (argon, etc.). First, 1.1 equivalents of an appropriate halogenating agent (SO₂Cl₂, Br₂, etc.) were dissolved in an appropriate amount of a suitable solvent such as 1,2 Dichloroethane (DCE). Subsequently, the solution was cooled to a temperature in the range of −15° C. to 0° C. The solution was then transferred dropwise to a solution or suspension containing 1 equivalent of a derivative of cystine and a suitable solvent such as DCE, where both the carboxylic acid groups and amino groups have been protected with suitable protecting groups (e.g., TFA, Cbz, Boc, Methyl ester, Ethyl ester, t-Butyl ester, etc.). The solution or suspension was also at a temperature of −15° C. to 0° C. The resulting mixture was then agitated for between 5 and 30 minutes and subsequently transferred dropwise to a suspension of potassium phthalimide (2 equivalents) in a suitable solvent such as DCE at −15° C. to 0° C. This resulting mixture was agitated at −15° C. to 0° C. for between 10 minutes and one hour and subsequently allowed to slowly warm to ambient temperature. The reaction was stopped when no further change appeared in the TLC analysis. The precipitate is removed either by filtration or centrifugation and the filtrate or supernatant was concentrated to yield the crude residue. The desired product was isolated by column chromatography and/or recrystallization. Any necessary deprotection was subsequently conducted and purification (if necessary) of the deprotected product was performed by column chromatography and/or recrystallization.

### EXAMPLE 2

### Synthesis of Aniline-Cysteine Derivatives

The reaction was performed entirely under anhydrous conditions and in an inert atmosphere (argon, etc.). First, 1.1 equivalents of an appropriate halogenating agent (SO₂Cl₂, Br₂, etc.) was dissolved in an appropriate amount of a suitable solvent such as 1,2 Dichloroethane (DCE). Subsequently, the solution was cooled to a temperature in the range of −15° C. to 0° C. The solution then was transferred dropwise to a solution or suspension containing one equivalent of a cystine derivative and a suitable solvent such as DCE, where both the carboxylic acid groups and amino groups have been protected with suitable protecting groups (TFA, Cbz, Boc, Methyl ester, Ethyl ester, t-Butyl ester, etc.). The solution or suspension also was at a temperature of −15° C. to 0° C. The resulting mixture was agitated for between 5 and 30 minutes and then was transferred dropwise to a solution containing four equivalents of aniline and a suitable solvent such as DCE at −15° C. to 0° C. The resulting mixture was agitated at −15° C. to 0° C. for between 10 minutes and one hour, and subsequently allowed to slowly warm to ambient temperature. The reaction was stopped when no further change appeared in the TLC analysis. The precipitate was removed either by filtration or centrifugation and the filtrate or supernatant was concentrated to give the crude residue. The desired product subsequently was isolated by column chromatography and/or recrystallization.

### EXAMPLE 3

### Synthesis of Phthalimide-Dithiocarbamate

The reaction was performed entirely under anhydrous conditions and in an inert atmosphere (argon, etc.). A solution of N-Bromo Phthalimide (1 equivalent) in DMF at 0° C. was added dropwise to a solution of Sodium Dithiocarbamate (1 equivalent) in DMF at 0° C. The mixture was agitated for one hour at 0° C. and then for one hour at 10-15° C. The reaction mixture was concentrated, the crude taken up into acetone and the precipitate filtered. The desired product was isolated by a combination of column chromatography and recrystallization.

### EXAMPLE 4

### Synthesis of Benzamide-Cysteine Derivatives

Initially, the sodium salt of benzamide was made in situ by reacting benzamide with sodium hydride in a suitable solvent such as tetrohydrofuran (THF). The reaction was allowed to proceed for 45 minutes at 0° C. followed by 30 minutes at ambient temperature. The sodium salt of benzamide was subsequently set aside under argon until needed in the reaction.

The reaction was performed entirely under anhydrous conditions and in an inert atmosphere (argon, etc.). First, 1.1 equivalents of an appropriate halogenating agent (e.g., SO₂Cl₂, Br₂, etc.) was dissolved in an appropriate amount of a suitable solvent such as 1,2 Dichloroethane (DCE). Subsequently, the solution was cooled to a temperature in the range of −15° C. to 0° C. The solution was then transferred dropwise to a solution or suspension containing one equivalent of the chosen derivative of cystine and a suitable solvent such as DCE, where both the carboxylic acid groups and amino groups have been protected with suitable protecting groups (e.g., TFA, Cbz, Boc, Methyl ester, Ethyl ester, t-Butyl ester, etc.). The solution or suspension also was at a temperature of −15° C. to 0° C. The resulting mixture was agitated for between 5 and 30 minutes and then was transferred dropwise to a suspension containing two equivalents of the sodium salt of benzamide and a suitable solvent such as DCE at −15° C. to 0° C. The resulting mixture was agitated at −15° C. to 0° C. for between 10 minutes and one hour, and subsequently allowed to slowly warm to ambient temperature. The reaction was stopped when no further change appeared in the TLC analysis. The precipitate was removed either by filtration or centrifugation and the filtrate or supernatant was concentrated to give the crude residue. The desired product subsequently was isolated by column chromatography and/or recrystallization. Any necessary deprotection then was conducted and purification (if necessary) of the deprotected product was performed by column chromatography and/or recrystallization.

### EXAMPLE 5

### Synthesis of Flutamide-Cysteine Derivatives

Initially, the sodium salt of flutamide was made in situ by reacting flutamide with sodium hydride in a suitable solvent such as tetrohydrofuran (THF). The reaction was allowed to proceed for 45 minutes at 0° C. followed by 30 minutes at ambient temperature. The sodium salt of flutamide was subsequently set aside under argon conditions until needed in the reaction.

The reaction was performed entirely under anhydrous conditions and in an inert atmosphere (argon, etc.). First, 1.1 equivalents of an appropriate halogenating agent (e.g., SO₂Cl₂, Br₂, etc.) was dissolved in an appropriate amount of a suitable solvent such as 1,2 Dichloroethane (DCE). Subsequently, the solution was cooled to a temperature in the range of −15° C. to 0° C. The solution was then transferred dropwise to a solution or suspension containing one equivalent of the chosen derivative of cysteine and a suitable solvent such as DCE, where both the carboxylic acid groups and amino groups have been protected with suitable protecting groups (e.g., TFA, Cbz, Boc, Methyl ester, Ethyl ester, t-Butyl ester, etc.). The solution or suspension also was at a temperature of −15° C. to 0° C. The resulting mixture was agitated for between 5 and 30 minutes and then was transferred dropwise to a suspension containing two equivalents of the sodium salt of flutamide and a suitable solvent such as DCE at −15° C. to 0° C. The resulting mixture was agitated at −15° C. to 0° C. for between 10 minutes and one hour, and subsequently allowed to slowly warm to ambient temperature. The reaction was stopped when no further change appeared in the TLC analysis. The precipitate was removed either by filtration or centrifugation and the filtrate or supernatant was concentrated to give the crude residue. The desired product subsequently was isolated by column chromatography and/or recrystallization. Any necessary deprotection then was conducted and purification (if necessary) of the deprotected product was performed by column chromatography and/or recrystallization.

### EXAMPLE 6

The following method was used to make N—S Bond-containing prodrugs from amide compounds such as, carbamazepine, acetanilide, flutamide, benzamides, N-methylbenzamide, phenylcarbamate and paclitaxel. This method is also applicable to amides that dissolve in THF.

Sulfuryl chloride (1 mmol) (1M solution in CH₂Cl₂) was added to pure diaryl/dialiphaticdisulfide (1 mmol) and cooled to 0° C. The resultant solution was stirred at 0° C. for 1 hour to yield a deep yellow solution of the aryl/aliphaticsulfenyl chloride. The cold (0° C.) aryl/aliphaticsulfenyl chloride generated above was transferred, via syringe, to a cold (0° C.) solution of the amide (1 mmol) containing Et₃N (1 mmol) in THF (5 mL). Immediately after addition, the appearance of a white precipitate was observed. This suspension was stirred at 0° C. for 1.5 hours. Water (2 mL) followed by ethyl acetate (10 mL) was added to the suspension and the layers were separated. The separated organic phase was dried over anhydrous Na₂SO₄ and evaporated in a rotavap below 40° C. to yield crude material. Flash column chromatography, using hexane/ethyl acetate as the eluent, of the crude material yielded the pure sulfenamide The percentage of each solvent used varied based on the polarity of the product.

### EXAMPLE 7

The following method was used to make N—S Bond-containing prodrugs from acetanilide. This method is also applicable to amides that dissolve in acetonitrile.

Sulfuryl chloride (1 mmol) (1M solution in CH₂Cl₂) was added to pure diaryl/dialiphaticdisulfide (1 mmol) and cooled to 0° C. The resultant solution was stirred at 0° C. for 1 hour to yield a deep yellow solution of the aryl/aliphaticsulfenyl chloride. The cold (0° C.) aryl/aliphaticsulfenyl chloride generated above was transferred, via syringe, to a cold (0° C.) solution of the amide (1 mmol) containing Et3N (1 mmol) in acetonitrile (5 mL). Immediately after addition, the appearance of a white precipitate was observed. This suspension was stirred at 0° C. for 1.5 hours. Water (2 mL) followed by ethyl acetate (10 mL) was added to the suspension and the layers were separated. The separated organic phase was dried over anhydrous Na2SO4 and evaporated in a rotavap below 40° C. to yield crude material. Flash column chromatography, using hexane/ethyl acetate as the eluent, of the crude material yielded the pure sulfenamide. The percentage of each solvent used varied based on the polarity of the product.

### EXAMPLE 8

The following method was used to make N—S Bond-containing prodrugs from linezolid. This method also is applicable to amides that do not readily dissolve in THF.

Sulfuryl chloride (1 mmol) (1M solution in CH₂Cl₂) was added to pure diaryl/dialiphaticdisulfide (1 mmol) and cooled to 0° C. The resultant solution was stirred at 0° C. for 1 hour to yield a deep yellow solution of the aryl/aliphaticsulfenyl chloride. The cold (0° C.) aryl/aliphaticsulfenyl chloride generated above was transferred, via syringe, to a cold (0° C.) solution of the amide (1 mmol) containing Et₃N (1 mmol) in a solvent mixture of dimethylformamide (DMF) (1 mL) tetrahydrofuran (THF) (10 ML). Immediately after addition, the appearance of a white precipitate was observed. This suspension was stirred at 0° C. for 1.5 hours. Water (2 mL) followed by ethyl acetate (10 mL) was added and the layers were separated. The separated organic phase was dried over anhydrous Na₂SO₄ and evaporated in a rotavap below 40° C. to yield a crude material. Flash column chromatography, using hexane/ethyl acetate was used as eluent, of the crude material yielded the pure sulfenamide. The percentage of each solvent used varied based on the polarity of the product.

### EXAMPLE 9

The following compound was synthesized using the method described in Example 6 and alternatively using the example described in Example 7. The compound is a prodrug of the parent N—H bond-containing pharmaceutical compound acetanilide.

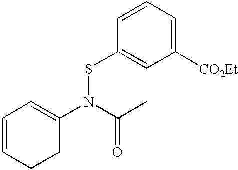

Separation by column chromatography on silica gel using hexane/ethyl acetate (4:1) as eluent gave a white solid (55%). ¹H NMR (400 MHz, CDCl₃): δ 1.38 (t, 3H.J=8 Hz) 2. 30 (s, 3H), 4.39 (q, 2H., J=8 Hz), 7.20-7.53 (series of m, 7H), 7.90 (m, 2H); ¹³C (100 MHz, CDCl₃): δ 61.3, 137.1, 144.9, 164.9; HRMS: calculated for [MH]⁺ C₁₇H₁₈NO₃S 316.1007; found 316.0993.

### EXAMPLE 10

A prodrug from parent N—H bond-containing pharmaceutical compound linezolid was synthesized using the method described in Example 8 above. Linezolid has the following properties: white crystalline solid, m.p.=180.1° C.; R_{f}=0 (eluent: 60% ethyl acetate/hexane)—polar; solubility in soybean oil ˜0.5 mg/ml- poorly soluble. The following is the structure of the parent N—H bond-containing pharmaceutical compound linezolid:

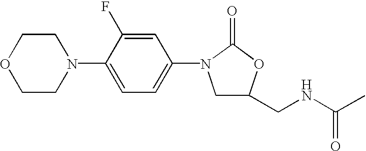

The following is the structure of the prodrug synthesized from the parent N—H bond-containing pharmaceutical compound linezolid.

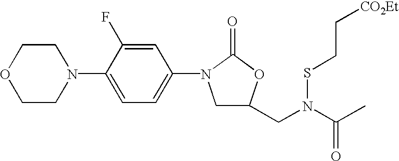

Separation by column chromatography on silica gel using hexane/ethyl acetate as eluent (3:2) gave a colorless viscous oil (72%). ¹H NMR (400 MHz, CDCl₃): δ 1.22 (t, 3H. J=8 Hz,), 2.39 (s, 3H), 2.65 (m, 2H), 2.98-3.25 (broad m, 4H), 3.68 (m, 2H), 3.88 (m, 4H), 3.99-4.19 (broad m, 6H), 4.86 (m, 1H), 6.90 (d d, 1H, J=2 Hz), 7.08 (d, 1H, J=2 Hz), 7.40 (d, 1H, J=6 Hz); ¹³C (100 MHz,CDCl₃): δ 23.1, 30.4, 38.1, 48.3, 51.5, 57.1, 60.0, 61.4, 67.3, 70.9, 107.5, 108.5, 114.1, 117.8, 133.2, 137.1, 154.7, 154.9, 156.8, 170.2, 177.1; HRMS: calculated for [MH]⁺ C₂₁H₂₈O₆N₃SF 470.1765; found 470.1735. The prodrug of this example had the following optimized properties: viscous oil; R_{f}=0.25 (eluent (60% ethyl acetate/hexane)—non-polar; solubility in soybean oil >30 mg/ml.

### EXAMPLE 11

A prodrug from parent N—H bond-containing pharmaceutical compound linezolid was synthesized using the method described in Example 8 above. The parent N—H bond-containing pharmaceutical compound linezolid had the following properties: white crystalline solid; m.p.=180.1° C.; R_{f}=0 (eluent: 60% ethyl acetate/hexane)—polar; and solubility in soybean oil ˜0.5 mg/ml—poorly soluble; and the following structure:

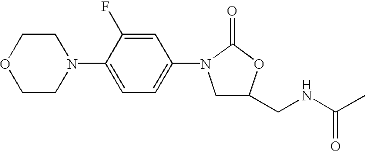

The prodrug synthesized from the parent N—H bond-containing pharmaceutical compound linezolid had the following structure:

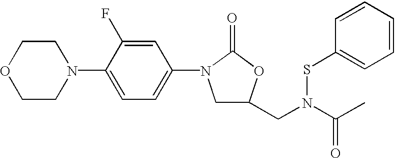

Separation by column chromatography on silica gel using hexane/ethyl acetate (3:2) as eluent (53%) gave a white crystalline solid. ¹H NMR (400 MHz, CDCl₃): δ 2.40 (m, 7H), 2.90 (m, 2H), 3.30 (m, 2H), 3.68 (m, 2H), 3.75-4.12 (m, 2H), 4.88 (m, 1H), 7.02 (m, 1H), 7.10-7.30 (m, 4H), 7.41 (m, 3H); HRMS: calculated for [MH]+ C22H25N3O4SF 466.1550; found 466.1545. The prodrug of this example had the following optimized properties: white crystalline solid; m.p.=120.9° C.; Rf=0.4 (eluent 60% ethyl acetate/hexane)—non-polar; solubility in soybean oil>10 mg/ml.

### EXAMPLE 12

The following compound was synthesized using the method described in Example 6 and is a prodrug of the parent N—H bond-containing pharmaceutical compound carbamazepine.

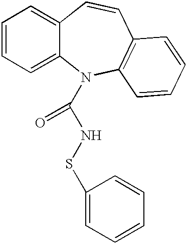

Separation by column chromatography on silica gel using hexane/ethyl acetate (4:1) as eluent (35%) provided a white solid. ¹H NMR (400 MHz, CDCl₃): δ 7.15-7.55 (series of m, 15H); 13C (100 MHz, CDCl₃): δ 134.5, 139.0, 139.8, 156.2; HRMS: calculated for [MH]+ 345.1062; found 345.1063.

### Example 13

The following compound was synthesized using the method described in Example 4 and is a prodrug of the parent N—H bond-containing pharmaceutical model compound benzamide.

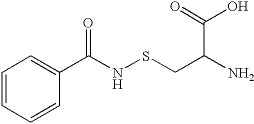

1HNMR (300 MHz, DMSO-d₆): □ 3.05 (d of d, 1H), 3.25 (d of d, 1H), 3.80 (broad), 4.15 (broad, 1H), 7.45 (t, 2H), 7.55 (t, 1H), 7.80 (d, 2H), 8.40 (broad), 9.60 (s, 1H). FAB+ 241.0 (M+1). Aqueous Stability: within a pH range of 4.0 to 7.0, the pH of maximum stability (projected by Eyring Plot) is 6.0 at 25 degrees Celsius with half life of −6.3 years (˜10 □g/mL benzamide-cysteine model prodrug solution in 35 mM Phosphate Buffer with Ionic Strength of 0.15M obtained by addition of NaCl). Pseudo-first order degradation kinetics were observed. When a 10 times molar excess of cysteine was added to the above solution, the model prodrug degraded (reverted to yield benzamide) with a half life of ˜20 to 30 minutes. In addition, the above benzamide-cysteine model prodrug had improved aqueous solubility compared to benzamide.

### EXAMPLE 14

The following compound was synthesized using the method described in Example 5 and is a prodrug of the parent N—H bond-containing pharmaceutical compound flutamide.

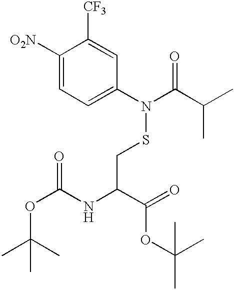

1HNMR (300 MHz, CDCl3): □ 1.20 (d, 6H), 1.35 (s, 9H), 1.45 (s, 9H), 3.05 (d of d, 1H), 3.25 (d of d, 1H), 3.50 (m, 1H), 4.35 (m, 1H), 5.20 (broad d, 1H), 7.60 (d of d, 1H), 7.75 (s, 11H), 7.90 (d, 1H).

### EXAMPLE 15

The following compound was synthesized using the method described in Example 1 and is a prodrug of the parent N—H bond-containing pharmaceutical model compound phthalimide.

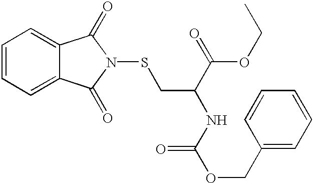

1HNMR (300 MHz, CDCl₃): □ 1.15 (t, 3H), 3.10 (d of d, 11H), 3.70 (d of d, 11H), 3.95 (m, 2H), 4.70 (m, 1H), 5.05 (d of d, 2H), 6.25 (d, 1H), 7.35 (broad s, 5H), 7.75 (m, 2H), 7.90 (m, 2H). Aqueous Stability: within a pH range of 3.5 to 5.0, the pH of maximum stability is 3.5 at 25 degrees Celsius with half life of ˜8 to 9 days (˜2 □g/mL phthalimide-cysteine model prodrug solution in 35 mM Acetate Buffer with Ionic Strength of 0.15M obtained by addition of NaCl). The aqueous degradation kinetics did not show pseudo-first order kinetics. When a 10 times molar excess of cysteine was added to the above solution, the model prodrug degraded (reverted to yield phthalimide) instantaneously (kinetics were not measurable).

### EXAMPLE 16

The following compound was synthesized using the method described in Example 6 and is a prodrug of the parent N—H bond-containing pharmaceutical compound carbamazepine

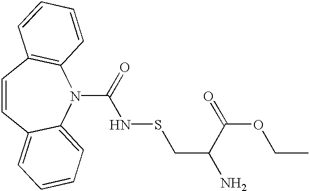

¹HNMR (300 MHz, DMSO-d₆): □ 1.15 (t, 3H), 2.85 (d of d, 1H), 3.05 (d of d, 1H), 4.05 (m, 1H), 4.15 (q, 2H), 6.95 (s, 2H), 7.10 (s, 1H), 7.35 (m, 2H), 7.40 (broad s, 6H), 8.40 (broad). FAB+ 384.1 (M+1). Aqueous Stability: within a pH range of 4.0 to 7.0, the pH of maximum stability (projected by Eyring Plot) is 4.0 at 25 degrees Celsius with half life of ˜181 days (˜2 □g/mL carbamazepine prodrug solution in 35 mM Acetate Buffer with Ionic Strength of 0.15M obtained by addition of NaCl). Pseudo-first order degradation kinetics were observed. In addition, the above carbamazepine prodrug had greatly improved aqueous solubility compared to carbamazepine.

Once the desired N—S bond-containing compound (i.e, prodrug) has been synthesized, it is placed in a suitable pharmaceutical carrier for delivery into the intended subject. Suitable pharmaceutical carriers for the present invention include any aqueous carriers which are useful for administering the pharmaceutical compounds or prodrugs thereof, preferably those which are non-toxic, otherwise inert, medically acceptable and compatible with the prodrug. Particularly useful are buffer saline based carriers. The present compositions may further comprise other active ingredients such as antimicrobial agents and other agents such as preservatives, solubilizers and stabilizers.

Once successfully delivered to the intended subject, the N—S bond-containing prodrug undergoes a reversion back to the original N—H bond-containing compound from which it was synthesized. Reversion may occur via hydrolysis or via exposure to compounds containing free sulfhydryl groups such as glutathione. Glutathione is present in human whole blood in a concentration of approximately 26.9 to 41.4 mg/100 ml with the mean value approximating 35.4 mg/100 ml. See Caren and Caren, Amer. J. Med. Sci., 221, 307 (1951). This concentration is sufficient to create reversion of a large variety of N—S bond-containing prodrugs back to the N—H bond-containing drug from which they were formed.

In conclusion, the present invention is directed to novel prodrugs of various pharmaceutical compounds containing one or more N—H bonds, as well as to methods of making and using such prodrugs. This unique utilization of N—S bond chemistry is applicable to the production of derivatives of various pharmaceutical compounds containing one or more N—H bonds and provides a general method for enhancing the chemical stability, solubility, cell membrane permeability, pharmacokinetic properties and other pharmaceutical properties of these classes of compounds, thus facilitating their clinical use. The present invention has been described in relation to particular embodiments which are intended in all respects to be illustrative rather than restrictive. Alternative embodiments will become apparent to those skilled in the art to which the present invention pertains without departing from its scope.

From the foregoing, it will be seen that this invention is one well adapted to attain all the ends and objects hereinabove set forth together with other advantages which are obvious and which are inherent to the prodrugs and methods of making and using such prodrugs herein disclosed.

Since many possible embodiments may be made of the invention without departing from the scope thereof, it is to be understood that all matter herein set forth or shown in the accompanying drawing is to be interpreted as illustrative and not in a limiting sense.

What the invention claimed is:

1. A compound having the following formula, 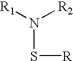
wherein R₁ and R₂ are residues of an N—H bond containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms;
and pharmaceutically acceptable salts thereof.

2. The compound of claim 1, wherein R₁ and R₂ may be the same or different and are one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

3. The compound of claim 1, wherein R has the following formula: 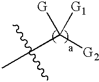
where a is an integer from 0-10 and
wherein a=0-10; wherein G, G₁, and G₂ may be the same or different and have the following formula: 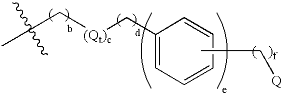
wherein b, c, d, e and f=0-10; wherein Q₁ is oxygen or sulfur and Q is selected from the group consisting of: 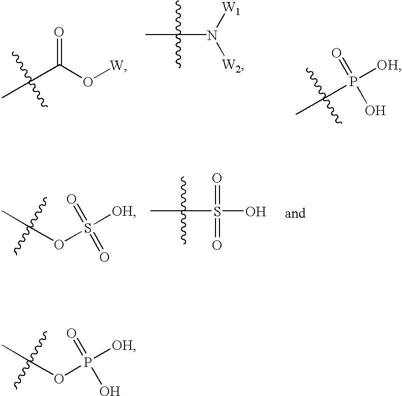
wherein W, W₁ and W₂ are selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

4. The compound of claim 1, wherein R is selected from the group consisting of:

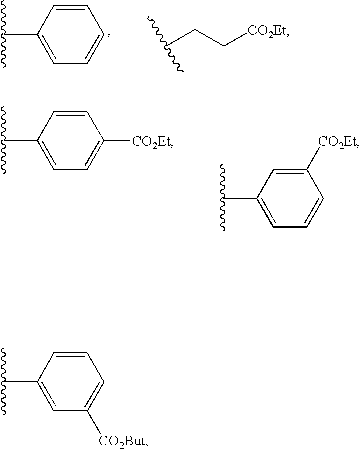

5. The compound of claim 1, wherein R is selected from the group consisting of: 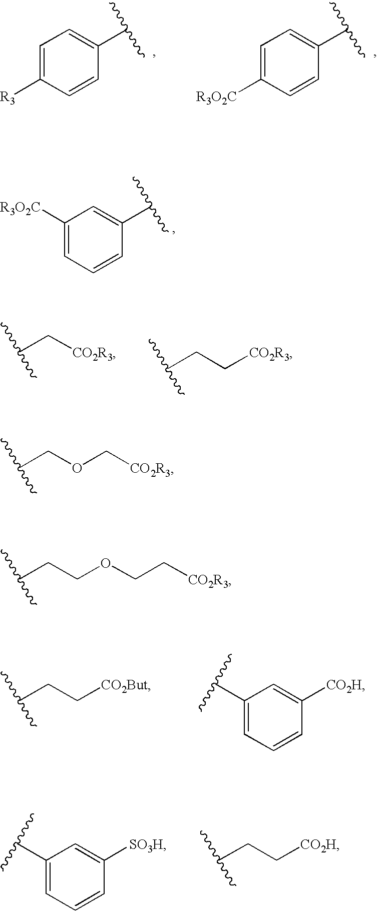
wherein R₃ is one of hydrogen, inorganic residue and an organic residue selected from the group consisting of:
a) straight-chain substituted or unsubstituted aliphatic groups, with or without any additional polar or non-polar functional groups and/or heteroatoms;
b) ranched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

6. The compound of claim 1, wherein the N—H bond-containing pharmaceutical compound is an imide.

7. The compound of claim 1, wherein the N—H bond-containing pharmaceutical compound is a hydantoin.

8. The compound of claim 1, wherein the N—H bond-containing pharmaceutical compound is uracil.

9. The compound of claim 1, wherein the N—H bond-containing pharmaceutical compound is a barbital.

10. The compound of claim 1, wherein the N—H bond-containing pharmaceutical compound is a amide.

11. The compound of claim 1, wherein the N—H bond-containing pharmaceutical compound is a urea.

12. The compound of claim 1, wherein the N—H bond-containing pharmaceutical compound is a carbamate.

13. The compound of claim 1, wherein the N—H bond-containing pharmaceutical compound is an amine.

14. The compound of claim 1, wherein the N—H bond-containing pharmaceutical compound is a heterocycle.

15. The compound of claim 1, wherein the N—H bond-containing pharmaceutical compound is a sulfonamide.

16. The compound of claim 1, wherein the N—H bond-containing pharmaceutical compound is a peptide.

17. The compound of claim 1, wherein the N—H bond-containing pharmaceutical compound is an oxazolidinone.

18. A method of using the prodrug of claim 1, to optimize stability, solubility, cell membrane permeability, pharmacokinetic properties and other pharmaceutical properties over the pharmaceutical compounds from which they are formed.

19. A method of administering the prodrug of claim 1.

20. The method of claim 19 wherein the method of administration is selected from the group consisting of parenteral, oral, intramuscular, subcutaneous, nasal, dermal, ophthalmic, inhalation, pulmonary, vaginal, rectal, aural and combinations thereof.

21. A pharmaceutical composition, comprising:
a compound according to claim 1; and a pharmaceutically acceptable carrier.

22. A compound having the following formula, 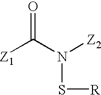
wherein Z₁ and Z₂ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms;
and pharmaceutically acceptable salts thereof.

23. A compound having the following formula, 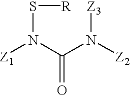
wherein Z₁, Z₂ and Z₃ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms; and pharmaceutically acceptable salts thereof.

24. A compound having the following formula, 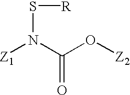
wherein Z₁ and Z₂ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms;
and pharmaceutically acceptable salts thereof.

25. A method of making a prodrug of an N—H bond-containing pharmaceutical compound comprising the reaction illustrated in Reaction Scheme I, 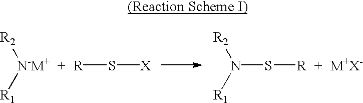
wherein M is a pharmaceutically acceptable organic or inorganic cation and X is any good leaving group,
wherein R₁ and R₂ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

26. A method of making a prodrug of an N—H bond-containing pharmaceutical compound comprising the reaction illustrated in Reaction Scheme II 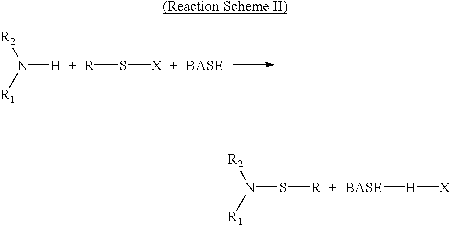
wherein X is any good leaving group,
wherein R₁ and R₂ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

27. A method of making a prodrug of an N—H bond-containing pharmaceutical compound comprising the reaction illustrated in Reaction Scheme III 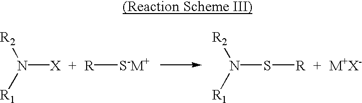
wherein M is a pharmaceutically acceptable organic or inorganic cation and X is any good leaving group,
wherein R₁ and R₂ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

28. A method of making a prodrug of an N—H bond-containing pharmaceutical compound comprising the reaction illustrated in Reaction Scheme IV 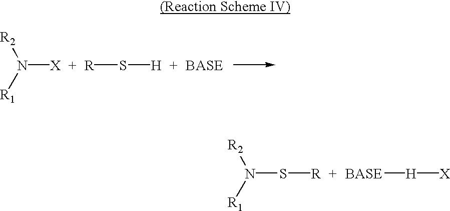
wherein X is any good leaving group,
wherein R₁ and R₂ are residues of an N—H bond-containing pharmaceutical compound, R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms.

29. An N—S prodrug having the following formula, 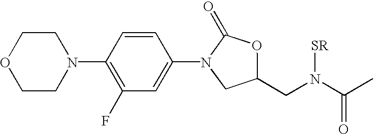
wherein R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms; and pharmaceutically acceptable salts thereof.

30. An N—S prodrug having the following formula, 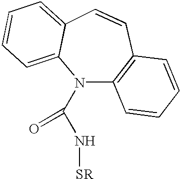
wherein R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms; and pharmaceutically acceptable salts thereof.

31. An N—S prodrug having the following formula, 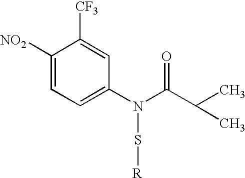
wherein R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms;
and pharmaceutically acceptable salts thereof.

32. An N—S prodrug having the following formula, 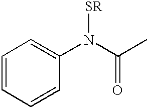
wherein R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
f) any combination of a), b), c), d) and e) with or without additional polar or non-polar functional groups and/or heteroatoms; and pharmaceutically acceptable salts thereof.

33. An N—S prodrug having the following formula, 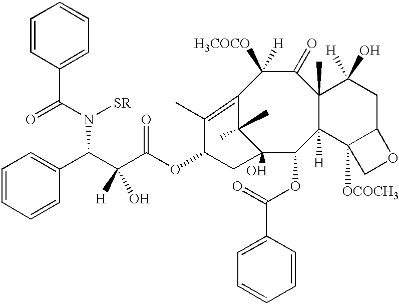
wherein R is one of a hydrogen, inorganic residue and an organic residue selected from the group consisting of:
a) straight-chain substituted or unsubstituted aliphatic groups, with or without any additional polar or non-polar functional groups and/or heteroatoms;
b) branched substituted or unsubstituted aliphatic groups, with or without any additional polar or non-polar functional groups and/or heteroatoms;
c) substituted or unsubstituted acyl groups, with or without any additional polar or non-polar functional groups and/or heteroatoms;
d) substituted or unsubstituted aromatic groups, with or without any additional polar or non-polar functional groups and/or heteroatoms;
e) substituted or unsubstituted cyclic groups, with or without any additional polar or non-polar functional groups and/or heteroatoms; and
d) any combination of a), b), c), d) and e) with or without additional polar or non-polar functional groups and/or heteroatoms;
and pharmaceutically acceptable salts thereof.
